(19) United States
(12) Patent Application Publication
(10) Pub. No.: US 2025/0012694 A1
(43) Pub. Date: Jan. 9, 2025

(54) MODULAR OPTICAL PROFILER FOR MEASURING LEVEL, OIL-WATER INTERFACE AND WATER CONTENT IN THE EMULSION LAYER

(71) Applicants: PETRÓLEO BRASILEIRO S.A. - PETROBRAS, Rio de Janeiro (BR); UNIVERSIDADE FEDERAL DO ESPIRITO SANTO — UFES, Vitória (BR)

(72) Inventors: LUIZ OCTAVIO VIEIRA PEREIRA, Rio de Janeiro (BR); WAGNER COIMBRA DE MORAES JUNIOR, Vitória (BR); ANSELMO FRIZERA NETO, Vitória (BR); ARNALDO GOMES LEAL JUNIOR, Vitória (BR)

(21) Appl. No.: 18/752,093

(22) Filed: Jun. 24, 2024

(30) Foreign Application Priority Data
Jul. 6, 2023 (BR) BR 1020230135900

(51) Int. Cl. G01N 13/00; G01N 33/28

(52) U.S. Cl. CPC G01N 13/00; G01N 33/2847

(57) ABSTRACT

The present invention relates to a modular optical profiler for measuring level, oil-water interface and water content in the emulsion layer. The profiler consists of optical sensor modules for temperature and pressure measurement and can be adapted for measurement in different tanks and operating processes, since it has small dimensions, flexibility and modularity. Furthermore, the profiler uses fiber optic Bragg gratings (FBG) to measure pressure and temperature gradients in production tanks. Further, the proposed modular optical profiler has the important advantage of measurement redundancy that, associated with the fusion of measurement data using different parameters, results in robust equipment that can function in different adverse operating conditions. Another important operational advantage is the possibility of customizing the number of sensors and their positions in the tank to adapt or optimize their operation in relation to the dimensional and operational characteristics of each tank or processing unit.

### FIELD OF THE INVENTION

The present invention pertains to the technical field of oil and gas, more specifically is related to monitoring storage, production or three-phase separator tanks onshore or offshore, and can be applied to the most diverse production platforms with the ability to distinguish between layers of water, oil and air/gas, and refers to a modular optical profiler for measuring level, oil-water interface and water content in the emulsion layer.

### BACKGROUNDS OF THE INVENTION

In the oil and gas industry, there is a need to measure the interface level between fluids in wells, tanks, reservoirs, processing vessels and storage vessels for fluids with different density, corrosivity and viscosity. Additionally, the processes in the oil and gas industry can contain high levels of pressure and temperature, which increases complexity and robustness requirements for instrumentation.

One of the main processes in the oil and gas industry is the separation of oil, water and gas. The hydrocarbons obtained from producing wells are a mixture of oil, gas, produced water and suspended solids. These different components are separated in the oil separation unit, which generally separates oil, gas and water through the difference in density of the immiscible fluids.

However, there are no well-defined, laminar layers for each of the fluids. Instead, there is an emulsion layer between the oil and water, which has its composition and behavior affected by the properties of the water and oil. In addition, there may be foam formation between the oil and gas layers in atmospheric tanks, which also have a dynamic behavior.

The foam and emulsion layer can introduce errors in the measurement of conventional interface level sensors. Additionally, sludge or wax may form on the tank walls, which may also influence the performance of the sensors.

In addition to the operational problems of measuring interface levels, caused by the formation of emulsion and foam layers, there are also limitations in current level measurement technologies related to the variety of fluids that can be inside an oil separator, in operation in harsh environments and safety issues.

Often, there is the presence of flammable gases in oil tanks, which can generate explosions. For this reason, the devices used in tank instrumentation cannot exceed certain voltage, current and capacitance limits.

When this occurs, the possible options for prolonging the shutdown are admitting that the unit will operate with a loss of revenue by reducing the load flow rate or removing the catalyst layer from the top of the reactor when the problem is concentrated in this region. The solution of using the most appropriate filters and catalyst gradient in loading is known and extensively used, but does not prevent the problem from occurring.

The possible unreliability in multi-interface level measurement systems leads to simplified control strategies in oil separators. This limitation in measuring the interface level leads companies to employ subsequent separators to achieve better separation of each phase of the fluid, which increases the cost of the plant and the complexity of maintenance.

In addition, if the separation between oil and water does not occur correctly, it is possible to obtain oil with a large amount of water in the refining process, which influences prices and the efficiency of oil processing. Water with oil above the specified level can contaminate the environment.

Specifically, for FPSO storage and separation tanks approximately thirty (30) meters long, there is no technology on the market for measuring the oil and water interface and measuring the water content of the emulsion layer.

At the same time, it is known that the advantages of fiber optic sensors and their wide use in industry make them an interesting option for interface level detection, especially when considering the intrinsic safety of these sensors, their flexibility and high data multiplexing/transmission capability. This last advantage of multiplexing capacity makes it possible not only to develop several sensors distributed on the same fiber optic cable to measure physical gradients, but also to develop sensors to measure different parameters using the same approach and data processing and acquisition systems.

Fiber optic sensors are innovative technologies that result in devices that are compact, lightweight, immune to electromagnetic fields, chemically stable and that allow multiplexing, that is, dozens of sensors can be used in the same fiber optic cable. In addition, they can be considered intrinsically safe, especially when compared to conventional electronic technologies. Fiber optic sensors have been used in industrial applications to measure various parameters, such as temperature, liquid level, acceleration, pressure, acoustic index and refractive index.

One of the main sensing devices used in fiber optics is the Fiber Bragg Grating (FBG), which consists of a periodic pattern of disturbance in the fiber's refractive index that allows the reflection of an optical signal of predefined wavelength and proportional to the period of the periodic disturbance and to the refractive index of the fiber optic. This reflected signal is intrinsically sensitive to the variations in the temperature of the medium and deformations applied to the fiber optic, since such parameters result in small variations in the period of disturbance of the refractive index that, in turn, results in small variations in the reflected wavelength, and such variations are proportional to the temperature or deformation applied to the fiber optic.

In view of the disclosure, and in order to solve the technical problems reported previously, the present invention describes a modular optical profiler that has the important advantage of measurement redundancy that, associated with the fusion of measurement data using different parameters, results in a robust equipment that can function in different adverse operating conditions. Another important operational advantage is the possibility of customizing the number of sensors and their positions in the tank to adapt or optimize their operation in relation to the dimensional and operational characteristics of each tank or processing unit.

The solution proposed in the present document uses fiber Bragg gratings (FBG) to measure pressure and temperature gradients in production tanks. Temperature dependence is a common limitation of some liquid level sensing technologies and can be overcome by having a temperature sensor next to each pressure sensor to compensate for the effects of temperature. The differences in thermal dynamics together with differences in density (which result in differences in hydrostatic pressure) result in the measurement of the water-oil interface level. Furthermore, the proposed approach is also capable of differentiating the levels of water, oil and emulsion in tanks with an accuracy of around 95% (obtained in the preliminary operational tests).

It is important to mention that the optical profiler consists of optical sensor modules for measuring temperature and pressure and can be adapted for measurement in different tanks and operating processes, since they have small dimensions, flexibility and modularity. These features make it possible not only to apply it at different measurement intervals (different tanks), but also allow for the possibility of customizing sensor performance parameters, such as resolution, sensitivity and even accuracy through optimizing the number of modules for each application. This technology delivers the position of the oil level, oil-water interface and water content in the oil layer, in a single solution. In the conventional approach, it would be necessary to install a capacitive type meter and microwave energy absorption meters together.

### STATE OF THE ART

Document BR1020210239786 is part of the general state of the art and describes the use of fiber optic sensors based on fiber Bragg gratings (FBGs) for measuring interface level using only temperature variations along the fiber optic. It has similarities related to only some of the components used in the sensor application, which are common to all FBG-based sensors, such as connectors, single-mode fibers and optical interrogator.

However, the present invention does not only use thermal variations, since they are sensitive to external environmental parameters and conditions, such as differences in solar radiation throughout the day and year and possible fluid inhomogeneity. In the case of the present invention, a multiparametric system (pressure and temperature) is proposed for analyzing the interface level, mainly including the hydrostatic pressure profile throughout the tank.

Such an approach makes it possible not only to measure the water-oil interface, but also to measure multiple interfaces in a tank in which the emulsion layer can be detected, resulting in measurements of water-emulsion-oil interfaces. Furthermore, the present invention is capable of measuring the BSW (acronym in English: Basic Sediments and Water), defined as the quotient between the flow rate of water added to the sediments being produced and the total flow rate of liquids from the emulsions through the responses of the multiple sensors.

In turn, the document Pereira et. al (2021), entitled “FBG-Based Temperature Sensors for Liquid Identification and Liquid Level Estimation via Random Forest” is part of the general state of the art and proposes a system using FBGs to measure liquid level using the temperature profile in the associated tank to artificial intelligence techniques. In this case, the system has similarities in the used components, such as connectors, single-mode fibers and optical interrogator, since they are common to all FBG sensor applications.

However, said document presents level measurement and identification of fluids considering only one fluid in the tank at a time; therefore, there is no water-oil interface level measurement as proposed in the present invention. Furthermore, the aforementioned document does not use the constructive form for measuring the density profile in the tank and is not capable of measuring multiple interfaces (water-emulsion-oil) nor the BSW of each solution, which differs substantially from the present invention in terms of technical difference and novelty.

Document U.S. Pat. No. 4,985,696, which is also part of the general state of the art, proposes a system for measuring the water-oil interface by resistivity. There are no constructive similarities or operating principles when compared to the present invention. It is important to mention that the aforementioned document presents a system that depends on electrical signals to carry out measurements that have major limitations for operation in classified areas, something that does not occur in the present invention in which only optical signals occur. It is also important to mention that (in contrast to the present invention) said document does not mention the measurement of multiple interfaces (water-emulsion-oil) nor the BSW of each solution.

Document CN102445253A also uses optical principles for interface level measurement, this being the only similarity with the present invention, since, in said document, the measurement is based on variations in the refractive index of the medium, which may result in technical problems of measurement due to dirt at the end of the fiber optic/collimator, which leads to an incorrect detection of the refractive index. Furthermore, there is no intrinsic multiplexing of sensors wherein several sensors can be multiplexed on the same fiber optic cable. The differences between the aforementioned document and the present invention are related to the measurement principle, since the present invention proposes the use of Bragg gratings in fiber optic. Furthermore, the differences are related not only to the analyzed parameters (pressure, density and temperature profiles in the present invention), but also to the possibility of measuring multiple interfaces (water-emulsion-oil) and the BSW of each solution.

Finally, document RU2328518C1, despite also using principles related to hydrostatic pressure for measuring the water-oil interface, comprises an operation that is fundamentally different from that proposed in the present invention. In the case of the invention in this document, there is a need to mark three control points that must be located at the limits of the oil and water columns, that is, the interface level (as well as the oil and water level) must be known a priori, which already constitutes an application completely different from that proposed in the present invention, wherein the levels of the multiple interfaces (water-emulsion-oil) can be measured in any position in the tank without any prior knowledge of their positions, through a modular system that can accommodate dozens of sensors that adapt to the different constructive aspects of production and storage tanks.

In this way, considering the disclosure above, the presence of a differential technical effect is noted considering not only the intrinsic advantages of FBGs sensors (i.e., intrinsic safety, multiplexing capacity and sustainability), but also differential technical effects related to density profile analysis that allow the measurement of multiple interfaces (water-emulsion-oil) and the BSW of each solution. In this case, the FBGs are incorporated into diaphragm elements for combined measurement of temperature and density profiles throughout the tank with an auxiliary temperature measurement system to provide redundancy and greater accuracy for the system when measuring.

### BRIEF DESCRIPTION OF THE INVENTION

The present invention relates to a modular optical profiler for measuring level, oil-water interface and water content in the emulsion layer. The profiler consists of optical sensor modules for temperature and pressure measurement and can be adapted for measurement in different tanks and operating processes, since it has small dimensions, flexibility and modularity. In addition, the profiler uses fiber optic Bragg gratings (FBG) to measure pressure and temperature gradients in production tanks. Furthermore, the proposed modular optical profiler has the important advantage of measurement redundancy that, associated with the fusion of measurement data using different parameters, results in robust equipment that can function in different adverse operating conditions. Another important operational advantage is the possibility of customizing the number of sensors and their positions in the tank to adapt or optimize their operation in relation to the dimensional and operational characteristics of each tank or processing unit.

### BRIEF DESCRIPTION OF THE FIGURES

To obtain a total and complete visualization of the object of this invention, the figures to which references are made are presented, as follows.

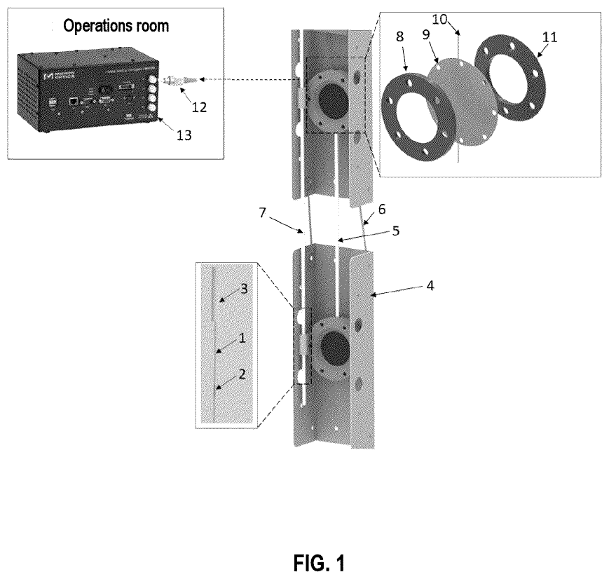

In FIG. 1, the proposed optical profiler (equipment) and the exploded view indicating each of the components are schematically represented.

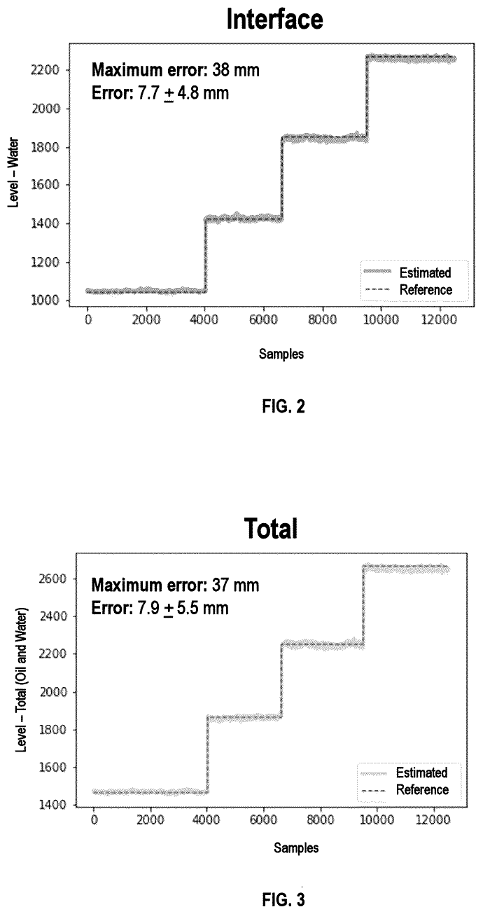

In FIG. 2, the water-oil interface level estimation results are schematically represented.

In FIG. 3, the estimation results of the total level (water and oil) estimated by the modular optical profiler are schematically represented.

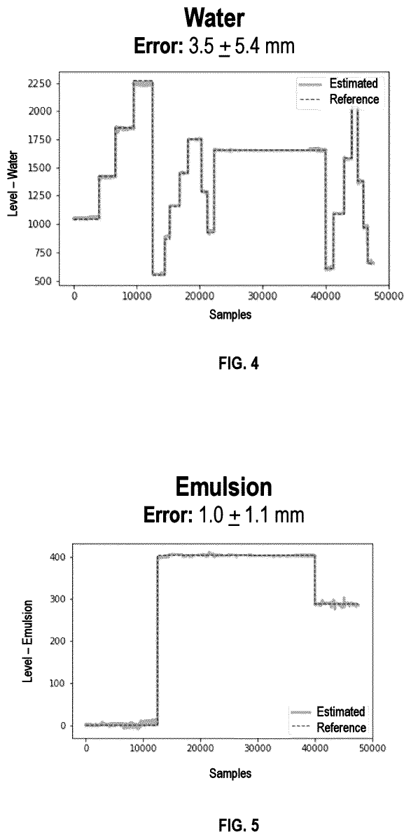

In FIG. 4, the results of the modular optical profiler for water level estimation are schematically represented.

In FIG. 5, the results of the modular optical profiler for emulsion level estimation are schematically represented.

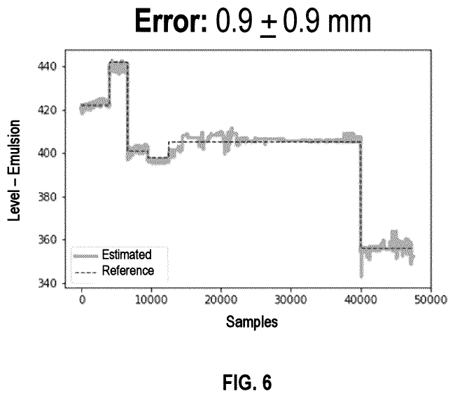

In FIG. 6, the results of the modular optical profiler for oil level estimation are schematically represented.

### DETAILED DESCRIPTION OF THE INVENTION

The present invention presents a modular optical profiler for measuring level, oil-water interface and water content in the emulsion layer. The profiler consists of optical sensor modules for temperature and pressure measurement and can be adapted for measurement in different tanks and operating processes, since it has small dimensions, flexibility and modularity.

In the constructive aspect of the invention, the main elements are the FBGs (1 and 10), which are engraved in single-mode fiber optics (2) from a beam of UV light that generates periodic disturbance with permanent changes in the index of refraction of the fiber optic in the region where it was applied. Therefore, it is possible to engrave dozens of FBGs in the same fiber optic, depending on the performance parameters desired for the interface level profiler.

The same stainless steel module (4) for fixing the optical profiler diaphragm has an FBG sensor for temperature measurement and another for pressure measurement, and, given the great multiplexing capacity of the sensors, it is possible to obtain dozens of connected modules each other. Considering the temperature measurement region, there is the single-mode fiber optic (1) with the engraved FBG in its core (2).

To isolate the temperature sensor from the effects of deformation and hydrostatic pressure, a stainless steel tubular protection (3) is positioned in the FBG region. Furthermore, the entire fiber optic is positioned inside a PTFE tube (7), for connecting temperature sensor modules and increasing mechanical strength and chemical inertness.

It is important to note that, although each module has a temperature sensor and a pressure sensor, it is possible to assemble several modules with the same fiber optic cable. The PTFE tube (7) is connected to the module (4) using clamps.

The module also has holes for positioning the diaphragm support screws (8 and 11). The front (8) and rear (11) supports are responsible for encapsulating the nitrile rubber diaphragm (9) in which a fiber optic with engraved FBG (10) or fiber optic with FBG embedded in the diaphragm (10) in its core is incorporated.

Similar to the FBG sensor for temperature measurement, the fiber optic cable with the pressure sensors (10) is positioned inside a PTFE tube (5) with the aim of increasing mechanical robustness and chemical inertness.

To increase the robustness of the connection between modules, stainless steel cables (6) are positioned to join consecutive modules without sacrificing flexibility between modules. Although FIG. 1 shows only two modules, the modular optical profiler is scalable to the point that it is possible to integrate a greater number of modules.

In order to connect the measuring means to the reading means, optical connectors (12) are used and connected to each channel of the optical interrogator (13), means of reading and acquiring the optical signal. The optical interrogator (13) consists of a broadband optical source, an optical circulator and a set of optical detectors that allow the user to be provided with the optical spectrum reflected from the sensor, that is, the necessary optical power and wavelength information for analyzing and processing of a signal from the sensors.

Given its great flexibility and customization capacity, the proposed invention can be applied to various storage, production or three-phase separator tanks onshore or offshore.

The intrinsic safety combined with the chemical inertness of these sensors (profiler) means that they can be applied to the most diverse production platforms with the ability to distinguish between layers of water, oil and air/gas.

Its application is possible in tanks that comprise practically all processes in the oil industry, mainly FPSO and terminal and refinery tanks, given the modularity, low weight and flexibility of the proposed sensor.

### EXAMPLES OF EMBODIMENT

The application and validation of the modular optical profiler was carried out using oil, brine and emulsion with different BSW with the profiler installed as presented in FIG. 1. In the carried-out tests, the optical profiler has six temperature and pressure measurement modules distributed in equidistant across the tank.

As previously discussed, the pressure sensors were FBGs vulcanized into nitrile rubber diaphragms (petroleum resistant), where the hydrostatic pressure leads to a deformation in the diaphragm that, in turn, results in a deformation proportional to the hydrostatic pressure in the optical sensor. Additionally, each module has an FBGs-based temperature sensor that has a fiber optic cable integrated into stainless steel tubes in PTFE cables for corrosion resistance and mechanical protection to hydrostatic pressures in the tank.

Accordingly, the optical temperature sensors are only sensitive to variations in this parameter. With the pressure and temperature data, an artificial neural network is applied to the results of each optical sensor (six pressure and six temperature sensors, in this case) to estimate the interface level or level of each fluid.

Different tests with variation of the water-oil interface level were carried out, while data from the temperature and pressure sensors were acquired by the Micron Optics sm125 optical interrogator at an acquisition frequency of 2 Hz, where the levels of each fluid were measured by a reference capacitive sensor, with the additional possibility of measurement through the tank's transparent sight glass.

Furthermore, additional tests consisted of positioning different levels of water, oil and emulsion to evaluate the modular optical profiler under these operating conditions. FIG. 2 presents a graph with the response of the optical sensors for measuring water and oil levels compared to the reference sensor installed in the process tank.

In this case, the results are presented as a function of the interface level (water level) and the total level (water level added to the oil level in these experiments). The results in FIG. 2 present the average errors (and standard deviation) for each curve, total level and interface level, where it is possible to notice an average measurement error below 8 mm. Considering the standard deviation, there can be noted that the system can present an accuracy of around 10 mm for measuring the water-oil interface in systems with only two phases.

To evaluate the performance of the modular optical profiler for cases with multiple interfaces, that is, a system composed of water, oil and emulsion, FIG. 3 presents a graph with the response of the sensors for measuring the levels of water, oil and emulsion. It is important to highlight that the capacitive sensor, used as a reference for analyzing systems with only water and oil, does not have the capacity to detect the level of water, oil and emulsion.

Accordingly, the comparison of the results of the modular optical profiler is carried out based on the data obtained by a measuring tape in each experimental condition. The results presented in FIG. 3 are separated into three curves: variation in water level, emulsion level and oil level.

In this case, there is an increase in the number of data that positively influences the performance of the data fusion algorithm, which results in a reduction in the measurement error of the sensors. Therefore, for tests with water, oil and emulsion, the modular optical profiler was able to estimate the level of each fluid in the tank with an average error of 3 mm, indicating the great accuracy of the proposed method.

Regarding the advantages of the present invention, economic and productivity advantages can be highlighted, wherein the sensors (profiler) have a lower cost than the main competing technologies (radar and radiation). In addition, there is the possibility of monitoring several tanks using the same system, resulting in greater dilution of the price per sensor.

Further, there are fewer elements in the sensor assembly, flexibility and customization capacity for different operating conditions thanks to the modularity of the profiler, which allows the same approach to be applied in different tanks (from 0.5 meters to more than 20 meters) changing only the number of profiler modules.

There are advantages related to health and safety, since the profiler comprises intrinsic safety, passive operation and galvanic protection. Furthermore, it is adapted for classified areas and with a smaller presence of employees in risk areas.

There are reliability advantages, wherein the profiler elements are resistant to oil, and also the proven ability to operate with more than 95% average accuracy. In addition, the profiler has redundancy of sensors and measurement techniques that guarantee its full functioning in adverse operating conditions.

There are environmental advantages, since the high accuracy combined with the low cost per sensor can result in less environmental damage due to the correct separation of water and oil. In addition, the operation has no environmental impact, with no potentially polluting elements. And the materials recycling technologies involved in sensor development are mature or in the process of maturing.

And finally, there are social advantages, as it is a new technology in the Brazilian market, with the possibility of generating new jobs in the technological sector and developing qualified labor.

Those skilled in the art will value the knowledge presented herein and will be able to reproduce the invention in the presented embodiments and in other variants encompassed by the scope of the attached claims.

1. A modular optical profiler for measuring level, oil-water interface and water content in the emulsion layer, configured to measure the level and/or interface in storage, production, or three-phase separator tanks onshore or offshore, the profiler comprising:
at least two fiber optic Bragg gratings, FBGs, which are engraved in single-mode fiber optics from a beam of UV light,
wherein the entire fiber optic is positioned inside a PTFE tube for connecting temperature sensor modules;
at least one perforated stainless steel module for fixing an optical profiler diaphragm,
wherein the at least one perforated stainless steel module has holes for positioning diaphragm support screws;
a stainless steel tubular protection positioned in a fiber optic Bragg gratings region,
wherein fiber optic cables with pressure sensors are positioned inside a PTFE tube;
at least two stainless steel cables positioned to join consecutive modules; and
at least two optical connectors connected to each channel of an optical interrogator.

2. The profiler according to claim 1, wherein the UV light generates a periodic disturbance with permanent changes in a refractive index of the fiber optic in a region to which it was applied.

3. The profiler according to claim 1, wherein the stainless steel module comprises a first FBG sensor for measuring temperature and a second FBG sensor for measuring pressure.

4. The profiler according to claim 1, wherein the stainless steel tubular protection is configured to isolates a temperature sensor from effects of deformation and hydrostatic pressure.

5. The profiler according to claim 1, wherein the first PTFE tube is connected to the at least one perforated stainless steel module using clamps.

6. The profiler according to claim 1, further comprising front and rear supports encapsulating a nitrile rubber diaphragm in which the fiber optic with engraved FBG is embedded in its core.

7. The profiler according to claim 1, wherein the optical interrogator consists of a broadband optical source, an optical circulator, and a set of optical detectors.

8. The profiler according to claim 1, wherein the optical interrogator provides a user with an optical spectrum reflected from a sensor with optical power and wavelength information.

9. The profiler according to claim 1, wherein the profiler is configured to distinguish between layers of water, oil, air/gas and emulsion and/or a combination thereof.

10. The profiler according to claim 1, wherein the storage, production, or three-phase separator tanks range from 0.5 meters to more than 20 meters.

11. The profiler according to claim 1, wherein an artificial neural network is applied to the result of each pressure sensor and/or temperature sensor module to estimate the interface level or the level of each fluid.

12. The profiler according to claim 1, wherein the profiler additionally measures a Basic Sediments and Water (BSW) of each solution.
